(19) United States
(12) Patent Application Publication
(10) Pub. No.: US 2014/0356854 A1
(43) Pub. Date: Dec. 4, 2014

(54) METHODS AND COMPOSITIONS RELATING TO MEVALONATE PHOSPHATE DECARBOXYLASE

(71) Applicant: Salk Institute for Biological Studies, La Jolla, CA (US)

(72) Inventors: Joseph P. Noel, San Diego, CA (US); Nikki M. Dellas, La Jolla, CA (US); Gerard Manning, San Diego, CA (US)

(21) Appl. No.: 14/295,905

(22) Filed: Jun. 4, 2014

Related U.S. Application Data
(60) Provisional application No. 61/831,093, filed on Jun. 4, 2013.

(51) Int. Cl. C12P 9/00; C12Q 1/527

(52) U.S. Cl. CPC C12P 9/00; C12Q 1/527
USPC 435/4; 435/131

(57) ABSTRACT

Methods and compositions related to a novel mevalonatephosphate decarboxylase enzymes are provided. The methods and compositions provided herein are particularly useful for the synthesis of isopentyl phosphate. The compositions and methods provided herein may also be used in combination with isopentyl phosphate kinase for the synthesis of isopentyl diphosphate.

### RELATED APPLICATIONS

This application claims the benefit of U.S. Provisional Application No. 61/831,093 filed Jun. 4, 2013, which is hereby incorporated in its entirety and for all purposes.

### BACKGROUND OF THE INVENTION

Isoprenoids constitute a large family of primary and secondary metabolites found in all three domains of life. These molecules play essential and specialized roles including modulation of membrane fluidity (cholesterol, hopanoids, squalene), defense and communication chemicals (mono-, sesqui- and diterpenes), photoprotection and energy transfer agents (carotenoids)(1), and growth regulating hormones (giberellins)(2). Additionally, isoprenoid quinones, chlorophyll, bacteriochlorophyll, and some cellular proteins are tethered to a polyisoprenoid chain to direct localization to membranes or to potentiate interactions with other biomolecules (3-5).

Isopentenyl diphosphate (IPP, 1) and its isomer, dimethylallyl diphosphate (DMAPP, 2), are the five carbon building blocks of all higher order isoprenoids. IPP is currently thought to be biosynthesized via one of two metabolic pathways: the mevalonate (MVA or MEV) pathway (6, 7) or the 1-deoxy-D-xylulose 5-phosphate (DXP) pathway (also known as the 2-C-methyl-D-erythritol 4-phosphate or MEP pathway)(8-10). These two pathways encode non-homologous enzymes that evolved independently to produce the same 5-carbon end product IPP (1); typically, a given organism uses one pathway or the other (11). Eukaryotes appear to encode the classical MVA pathway (with some exceptions, see (12)); plants additionally encode the DXP pathway that functions in the chloroplast. Most bacteria employ the DXP pathway with the exception of several groups that contain full or partial MVA pathway genes (13, 14).

Peculiarly, archaea do not encode gene homologs for the DXP pathway and most encode an incomplete classical MVA pathway (lacking identifiable genes encoding enzymes for one or both of the last steps). This observation is puzzling due to the essentiality of isoprenoid compounds in archaeal metabolism, particularly in cell membrane biosynthesis. Recent phylogenetic and experimental data suggest that archaea encode an alternative MVA pathway, which bifurcates from the classical pathway following the synthesis of phosphomevalonate (MVAP, also known as mevalonate 5-phosphate, 3) (15-17) (FIG. 1). The classical MVA pathway first phosphorylates MVAP (3) and then decarboxylates diphosphomevalonate (MVAPP, also known as mevalonate 5-diphosphate, 5) using phosphomevalonate kinase (PMK) and mevalonate 5-diphosphate decarboxylase (MDD, also known as MDC or DPM-DC), respectively. The alternative MVA pathway is posited to reverse these steps; decarboxylation of MVAP (3) followed by phosphorylation of isopentenyl phosphate (IP, 4) (FIG. 1). The first step requires a currently undetected enzyme activity, MVAP decarboxylase (MPD), to decarboxylate MVAP (3) to IP (4). The final phosphorylation activity is a well-characterized reaction performed by isopentenyl phosphate kinase (IPK), an ATP-dependent kinase that phosphorylates IP (4) forming the essential five-carbon building block, IPP (1) (FIG. 1).

### BRIEF SUMMARY OF THE INVENTION

In one aspect, a method of synthesizing an isopentyl phosphate or analog thereof is provided. The method includes contacting a phosphomevalonate (MVAP) or analog thereof with a mevalonatephosphate decarboxylase (MPD)-like mevalonate 5-diphosphate decarboxylase (MDD) enzyme, thereby forming an isopentyl phosphate or analog thereof, wherein the MPD-like MDD enzyme lacks detectable MDD activity.

In another aspect, a method of synthesizing an isopentyl diphosphate or analog thereof is provided. The method includes contacting a phosphomevalonate (MVAP) or analog thereof with a mevalonatephosphate decarboxylase (MPD)-like mevalonate 5-diphosphate decarboxylase (MDD) enzyme, thereby forming an isopentyl phosphate or analog thereof. The method further includes contacting the isopentyl phosphate or analog thereof and a phosphate or phosphate analog donor with an isopentenyl phosphate kinase or an mevalonate 5-diphosphate decarboxylase, thereby forming an isopentyl diphosphate or analog thereof.

In another aspect, a method of identifying an amino acid substitution within a mevalonatephosphate decarboxylase (MPD)-like mevalonate 5-diphosphate decarboxylase (MDD) enzyme that increases isopentyl phosphate formation rate is provided. The method includes determining a hypothetical binding position of a phosphomevalonate (MVAP) within an active site of a first MPD-like MDD enzyme using a computer modeling program. Based on the hypothetical binding position a test mutated MPD-like MDD enzyme including an amino acid substitution relative to the first MPD-like MDD enzyme is made. The test mutated MPD-like MDD enzyme is contacted with an MVAP and determining a first rate of formation of an isopentyl phosphate. The first rate of formation of the isopentyl phosphate is compared with a second rate of formation, wherein the second rate of formation is determined by contacting the first MPD-like MDD enzyme and the MVAP, wherein a higher first rate of formation relative to the second rate of formation indicates the amino acid substitution increases isopentyl phosphate formation rate.

### BRIEF DESCRIPTION OF THE DRAWINGS

FIG. 1. The classical and alternative MVA Pathways. Both branches of the MVA pathway begin with acetyl CoA (and acetoacetyl CoA) and proceed through a series of enzymatic reactions involving 3-hydroxy-3-methylglutary-CoA Synthase (HMGS), 3-hydroxy-3-methylglutary-CoA Reductase (HMGR, the rate-limiting step), and mevalonate kinase (MVK) before branching. At the bifurcation, the canonical MVA pathway guides phosphomevalonate through an additional phosphorylation and subsequent decarboxylation carried out by phosphomevalonate kinase (PMK) and diphosphomevalonate decarboxylase (MDD), respectively. The alternative MVA pathway hypothetically decarboxylates phosphomevalonate prior to the phosphorylation reaction carried out by IPK (16). The enzymes MVK, PMK, MDD, and IPK all consume ATP during catalysis.

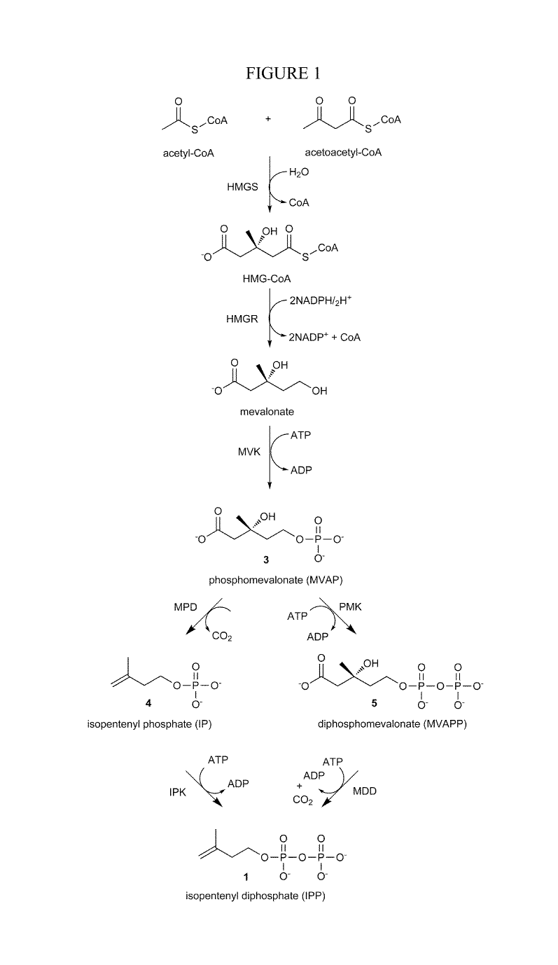

FIG. 2. Phylogenetic distribution of IPK among the three domains of life. Maximum likelihood tree of selected IPK protein sequences. Eukaryotes are highlighted with blues, selected archaeal clades with grays and a small group of bacteria with purple. The tree is anchored by several bacterial fosfomycin kinases.

FIG. 3. In vitro reconstruction and GC-MS analysis of the alternative and classical MVA pathways. (a) In the late MVA pathway, enzymes of the alternative (orange) and classical (blue) pathways are depicted as arrows. The y-axis of the graph represents combinations of enzymes from either of these two pathways that yield the end product, 5-epi-aristolochene (5-EA). Abbreviations of organisms are as follows: Bf=Branchiostoma floridae, At=Arabidopsis thaliana, Ss=Sulfolobus solfataricus, and Rc=Roseiflexus castenholzii. Note, RcMPD is colored both orange and blue on the y-axis, depending on whether it is being tested as an MDD (blue) or an MPD (orange). (b) In vitro assays include either the alternative or the classical enzymes to produce IPP (1) (as shown in panel a) as well as all downstream enzymes, including isopentenyl phosphate isomerase (IPPI) to produce DMAPP, farnesyl diphosphate synthase (FPPS) to produce farnesyl diphosphate (FPP) and tobacco 5-epi-aristolochene synthase (TEAS) to produce the sesquiterpene product, 5-EA (detected and quantified by GC-MS).

FIG. 4. Structural comparisons between canonical MDDs and MPD-like MDDs. (a) Interactions between the terminal phosphate of 6F-MVAPP and the surrounding amino acid residues from the crystal structure of S. epidermis MDD (left, PDB ID 3QT7(31)) and the 3D model of MPD from R. castenholzii (right). S. epidermis MDD has multiple interactions with the diphosphate of MVAPP compared to R. castenholzii MPD. (b) Interactions between the monophosphate of modeled 6F-MVAP and the surrounding amino acids in a superposition of the modeled R. castenholzii MPD on the crystal structure of S. epidermis MDD. In R. castenholzii MPD, two new sides chains, Arg83 and Lys161, provide additional electrostatic interactions with the monophosphate group of MVAP. These amino acids would also sterically clash with the terminal phosphate of MVAPP if bound, indicating that the active site topology of R. castenholzii MPD facilitates substrate recognition of MVAP and not MVAPP.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butyryl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)N R′R″, —NR″C(O)R′, —NR′—C(O)NR″R′″, —NR″C(O)₂R′, —NR—C(NR′R″R′″)=NR″″, —NR—C(NR′R″)═NR′″, —S(O)R′, —S(O)₂R′, —S(O)₂NR′R″, —NRSO₂R′, —CN, and —NO₂ in a number ranging from zero to (2 m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC (O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from:
      oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., National Center for Biotechnology Information [NCBI] web site or the like). Such sequences are then said to be “substantially identical.” As described below, the preferred algorithms can account for gaps and the like. Identity may exist over a region that is at least about 25 amino acids or nucleotides in length, or over a region that is 50-250 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the NCBI. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

For PCR (polymerase chain reaction), a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec—2 min., an annealing phase lasting 30 sec.—2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

### II. Examples

Applicants performed an extensive phylogenetic analysis to reveal a spotty distribution of IPK-bearing organisms in animals, several fungi, all plants, and some bacteria. Following purification and characterization of several representatives from each domain of life, Applicants show that fully active and highly specific IPKs unexpectedly exist outside of Archaea. Applicants also overexpressed, purified, and characterized MDDs and PMKs from these same organisms to assess the biochemical function of each.

It is noteworthy that certain archaeal and bacterial groups contain components of both the classical and alternative MVA pathways identified through sequence homology. Archaeal species from the family Halobacteriaceae and the class Thermoplasmata and bacterial species from the class Chloroflexi contain genes from both the alternative (IPK) and classical (MDD) MVA pathways, but are missing one gene from each branch (PMK and MPD, respectively)(14). It is surprising that two incomplete branches of the MVA pathway exist in one organism given the essentiality of the MVA pathway in the absence of the DXP pathway. Upon functional characterization of these enzymes from the Chloroflexi bacterium, Roseiflexus castenholzii (lacking the DXP pathway), Applicants biochemically demonstrate that a functional alternative MVA pathway does exist. In R. castenholzii, the putative MDD of the classical MVA pathway unexpectedly encodes a functional MPD associated with the alternative MVA or lost pathway. Moreover, the IPK-like gene encodes a functional IPK, thereby uncovering the first definitive experimental evidence for a fully operative alternative MVA pathway in nature.

Results

Phylogenetic Analysis of IPK Homologs Across all Three Domains of Life.

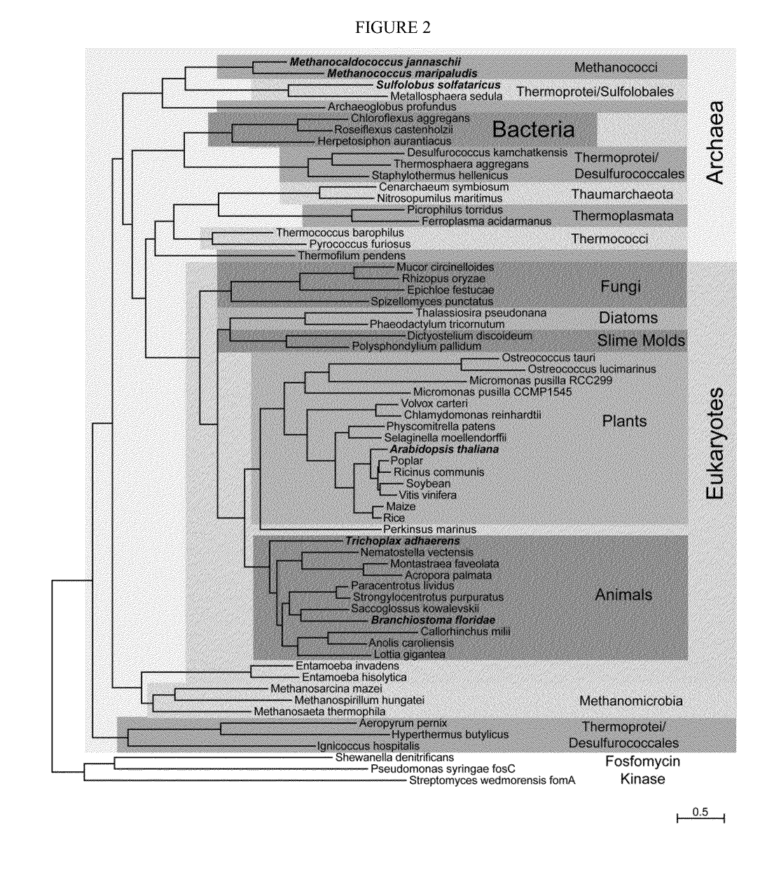

IPK is a member of the amino acid kinase (AAK) superfamily. To date, the search for eukaryotic IPK-like genes has been limited since putative eukaryotic IPK homologs are divergent in sequence and can be difficult to distinguish from other AAK superfamily genes (14). Within IPK, an active site histidine residue, not conserved among AAK family members, was found to be uniquely responsible for IP binding and catalysis (18). With this structurally and functionally defined residue as an indication of IPK activity (18), Applicants identified IPK homologs from all three domains of life. Applicants used PSI-BLAST and profile Hidden Markov Models (HMMs) to detect IPK homologs in public protein, expressed sequence tag (EST), and genome databases. IPK homologs appear in almost all archaea (14, 15), a small cluster of Chloroflexi bacteria (14), every sequenced green plant genome, and, in an exceptionally sporadic distribution, across most major eukaryotic lineages (FIG. 2).

IPK appears to have been lost independently in many animal lineages (FIG. 2). It is absent from choanoflagellates and sponges, but found in early branching animals such as Trichoplax, cnidarians (Nematostella, but not Hydra), and corals. It is found in bilaterians, including molluscs (Aplysia sp., Lottia giganteas), annelids (earthworm and leech), and a crustacean (lobster), but not in any insect or nematode. Within deuterostomes, it is found in the sea urchin and sea star, as well as the hemichordate Saccoglossus kowalevskii (acorn worm) and the chordate Branchiostoma floridae (lancelet). Within the vertebrates, it is found in a shark (Callorhinchus milii) but no teleost fish; in an amphibian (the newt Notophthalmus viridescens) but not in frogs; and in a lizard (Anolis carolinensis) and a snake (Philodryas olfersii), but not in any bird or mammal. Within the fungi, IPK is present in one of two sequenced chytrids and two other basal fungal genomes, but is otherwise absent.

While all identified IPK homologs retain 11 invariant residues (see Table 1) their sequences are otherwise highly divergent, and their predicted phylogeny agrees only in part with organismal taxonomy. For instance, while all plants and animals cluster well, sequences from the archaeal class Thermoprotei fall into four distinct clusters on the calculated phylogenetic tree, and the eukaryotic genus Entamoeba sequences cluster with the archaeal class Methanomicrobia (FIG. 2). This sporadic clustering pattern suggests either horizontal gene transfer, or ongoing functional shifts between orthologs in terms of substrate specificity, activity, or other physiological functions.

Kinetic Characterization of IPKs, PMKs, and MDDs from the Three Domains of Life.

Applicants overexpressed, purified, and characterized seven IPK homologs selected from all three domains of life in order to determine which enzymes are bona fide IPKs. These include the previously characterized IPK from M. jannaschii (16, 18) and homologs from two other archaea (Methanococcus maripaludis and Sulfolobus solfataricus), a bacterium (Roseiflexus castenholzii), and three eukaryotes: Trichoplax adhaerens (early-branching metazoan), Branchiostoma floridae (chordate), and Arabidopsis thaliana (plant). Kinetic experiments performed using a lactate dehydrogenase-pyruvate kinase coupled assay demonstrate that all seven IPK homologs catalyze the phosphorylation of IP (4) to IPP (1); kinetic constants are reported in Table 2. In summary, their steady-state catalytic efficiencies range from 0.04-2.4 s⁻¹ μM⁻¹, with k_{cat} values from 0.91-27.2 s⁻¹ and K_{M} values from 23.6-0.79 μM.

It is noteworthy that many of these IPK-bearing organisms also appear to encode annotated genes associated with the classical MVA pathway. To explore possible pathway redundancy, Applicants overexpressed, purified, and characterized PMKs and MDDs from organisms containing biochemically verified IPKs, including the animal, B. floridae, the plant A. thaliana, the archaeon S. solfataricus (one of the few archaea that encodes a complete classical MVA pathway), and the bacterium R. castenholzii (an organism that encodes MDD but no obvious PMK). PMKs from B. floridae and A. thaliana are active and are catalytically similar (Table 2). While PMK from S. solfataricus is active, its MDD decarboxylates MVAPP (5) at a very slow rate; under steady state conditions, Applicants were unable to accurately assign kinetic parameters for this enzyme.

Unexpectedly, the MDD homolog from R. castenholzii does not catalyze the decarboxylation of MVAPP (5) to IPP (1) at a measurable rate. Instead, it behaves as an MPD and catalyzes the decarboxylation of MVAP (3) to IP (4) with a k_{cat} of 0.94 s⁻¹ and an apparent K_{M} for MVAP of 1.150 mM (Table 2). This enzyme, by sequence homologous to MDD, has acquired an unexpected new function as a bona fide MPD playing the role of the long sought MPD of the alternative MVA pathway. To the best of Applicants'knowledge, this is the first newly discovered enzyme functioning as an MPD, and further suggests that functional expectations in the canonical MVA biosynthetic pathway, based upon gene homology alone, must be reexamined.

GC-MS quantification of fully functional alternative and classical MVA pathways. To confirm that the functional R. castenholzii MPD (MDD-like protein sequence) catalyzes the transformation of MVAP (3) to IP (4) in a reconstituted in vitro pathway, a five-enzyme in vitro reaction mixture was assembled. Briefly, MVAP (3) and ATP were incubated for 30 min with MPD and IPK from R. castenholzii. The reaction mixture was then diluted into a mixture containing all necessary downstream enzymes to biosynthesize the GC-detectable sesquiterpene 5-epi-aristolochene (5-EA, 6). Similar reactions were performed for the in vitro reconstitution of the classical MVA pathway using PMK and MDD from each respective organism.

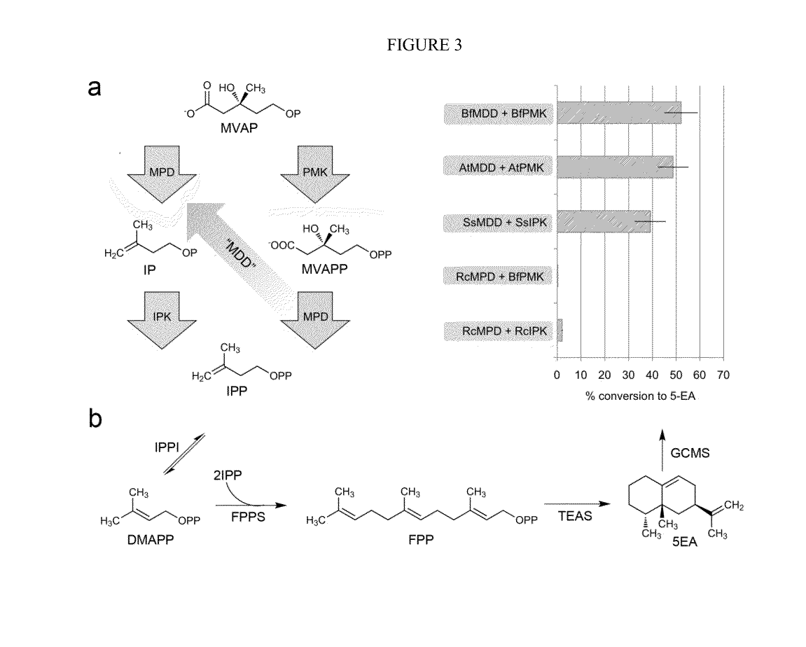

The in vitro classical MVA pathway reactions for S. solfataricus, A. thaliana, and B. floridae yield 39.2%, 48.7%, and 52.1% conversion of MVAP (3) to 5-EA (6), respectively (FIG. 3). The in vitro classical MVA pathway reaction from R. castenholzii (using B. floridae PMK to complete the missing step) yields less then 0.2% conversion to 5-EA (FIG. 3). In contrast, the in vitro alternative MVA pathway from R. castenholzii enzymes yields a 2.0% conversion of MVAP (3) to 5-EA (6). B. floridae, A. thaliana, and S. solfataricus were tested for a possible alternative MVA pathway components using their IPKs and MDDs. These reactions do not yield any detectable 5-EA (6), indicating that these MDDs behave as expected from their annotations and lack detectable MPD activity. Control reactions lacking ATP, enzyme, or MVAP (3) do not yield any 5-EA (6).

Molecular modeling of the MDD-like MPD from R. castenholzii reveals an altered terminal phosphate binding site. The experiments described above indicate that the MDD-like enzyme from R. castenholzii does not accept MVAPP (5) as a substrate, and therefore, lacks detectable MDD activity. Instead, it possesses a newly discovered biochemical activity, use of MVAP (3) as a substrate for the decarboxylation of MVAP (3) to IP (4). These biochemical results conclusively demonstrate that this MDD-like gene in fact encodes an MPD. A closer examination of sequences and three-dimensional structures of MDDs highlights catalytic elements that set MPD from R. castenholzii apart from other bacterial MDDs. A structural model of MPD from R. castenholzii was computed using Swissmodel (28, 29) and the Staphylococcus aureus MDD structure (PDBID 2HK2) as a template (26% sequence identity to MPD from R. castenholzii)(30).

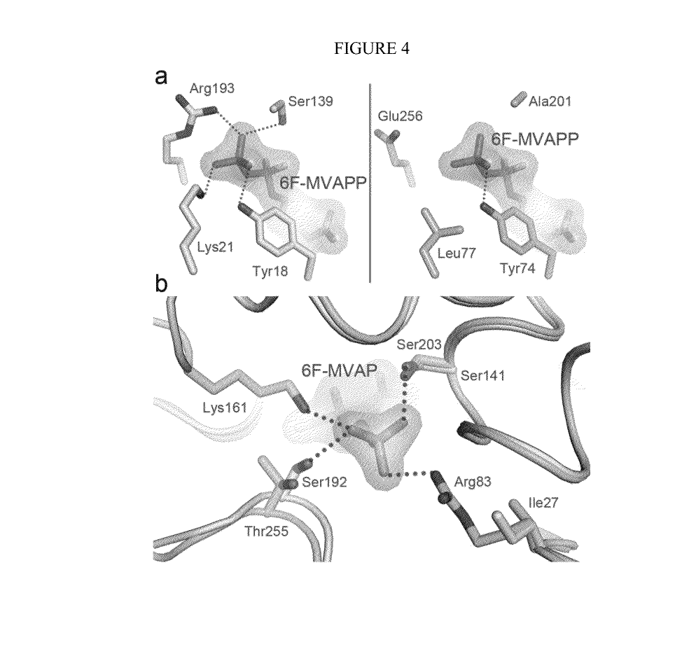

Residues surrounding the diphosphate group of 6F-MVAPP (5) bound to S. epidermis MDD (PDBID 3QT7(31)) were then contrasted with the corresponding residues in the R. castenholzii MPD model. A comparison of the phosphate-binding residues of the canonical MDD from S. epidermis to the same positions in the R. castenholzii MDD-like MPD reveals that the R. castenholzii MPD lacks several conserved hydrogen bonding interactions associated with recognition of the terminal phosphate of MVAPP's (5) diphosphate moiety (FIG. 4a). Second, the R. castenholzii MPD contains additional residues including arginine and lysine side chains that appear poised to bind the single phosphate of MVAP (3). Finally, these residues, unique to atypical MDDs such as the R. castenholzii MDD-like MPD, clash sterically with the terminal phosphate of MVAPP's (5) diphosphate (FIG. 4b, Table 3). Phylogenetic analysis shows that MDDs from the Chloroflexi are distant from those of other bacteria, but similar to those of the two archaeal groups that also encode an IPK but no PMK, the Haloarchaea and Thermoplasmata. The model described above illustrates a logical route through which atypical MDDs may have lost MVAPP (5) substrate selectivity and acquired preferential activity against MVAP (3).

Discussion

The Chloroflexi are considered to be one of the oldest phyla of photosynthetic organisms (32, 33). As photoheterotrophs, Chloroflexi use light for energy but can not fix carbon dioxide as their primary source of carbon (34). Of the six bacterial phyla reported to contain MVA pathway-bearing organisms, only the Chloroflexi contain IPK (14); however, they lack an obvious PMK. Although it appears from genome annotations that the Chloroflexi encode two broken branches of the MVA, Applicants demonstrated that the MDD-like protein in fact encodes a functional MPD. MPD and IPK from the Chloroflexi bacterium, R. castenholzii, assemble an unforeseen alternative MVA pathway that catalyzes sequential MVAP (3) decarboxylation and IP (4) phosphorylation reactions forming the essential isoprenoid building block IPP (1).

The neofunctionalization of the MDD gene to a MPD gene in R. castenholzii appears logical given the predicted active site topology of the MDD-like MPD compared to canonical MDDs particularly in the regions surrounding the diphosphate moiety of MVAPP (5) in canonical MDDs and the monophosphate group of MVAP (3). The majority of the residues surrounding the diphosphate of MVAPP (5) in canonical MDDs are strictly conserved in structure- and sequence-based analyses (30). On the other hand, archaeal MDDs from Haloarchaea and Thermoplasmata contain MPD-like active site residues associated with MVAP (3) binding and turnover and the protein sequences cluster with the MDD-like MPD from Chloroflexi (Table 3)(14). These are also the two archaeal groups that encode IPK but not PMK, suggesting that they also use this assembly of the alternative MVA pathway.

While archaea contain an active IPK consistent with the presence of the alternative MVA pathway, most lack typical or atypical MDD genes. S. solfataricus and other species from the archaeal order Sulfolobales are the exception. In Applicants'experiments, S. solfataricus PMK and MDD activities were detected in steady state kinetic experiments and GC-MS assays, respectively (Table 2, FIG. 3). A recent publication on the characterization of the classical MVA pathway enzymes in S. solfataricus supports an active classical MVA pathway, consistent with Applicants'in vitro results (35). Notably, while Applicants'experiments demonstrate that IPK is active in an in vitro assay, their experiments involving cell-free extracts of S. solfataricus indicate that IPK activity is undetectable. Therefore, if archaea do encode an alternative MVA pathway, a gene serving the decarboxylase function has yet to be identified.

One candidate gene suggested to encode the necessary decarboxylation activity is an uncharacterized dioxygenase-like gene that resides within the MVA operon in most Archaea (MJ0403 in in M. jannaschii)(16). MJ0403 encodes a protein that is similar in sequence to subunit B of class III extradiol ring-cleavage dioxygenases and is also homologous to the human protein MEMO (mediator of erbB2-driven cell motility). Thus far, Applicants' in vitro attempts to demonstrate decarboxylase activity for MJ0403 have failed to show any turnover of MVAP.

Eukaryote genomes examined to date appear to encode a classical MVA pathway with few exceptions (see (12)). Nevertheless, Applicants identified a spotty distribution of eukaryotes that also contain IPK, a gene associated with an alternative route to IPP (1) through the MVA pathway. Since many eukaryotes encode putative enzymes of unknown function, they may also encode a cryptic MPD that would serve in an alternative biosynthetic route to IPP (1). All IPKs tested thus far were fully functional, demonstrating that true IPKs persist across most eukaryotic lineages, while some have been lost during rare evolutionary events, probably due to partial redundancy with the MVA pathway. The unusual phylogeny of IPK coupled with its membership in a family of kinases that phosphorylate such a broad range of substrates leave open the alternative possibility that IPK may assume varied physiological roles, including phosphorylation of an IP-like substrate or IP recycling (that may accumulate in vivo as a consequence of phosphatase-dependent IPP degradation to IP).

While the IPK gene is present within a spotty distribution of eukaryotes, the gene appears to be universally retained across the green plant lineage, which suggests that it plays a more universal role within the plant kingdom. Plants are unique in that they encode two IPP-synthesizing pathways, the DXP pathway and the MVA pathway, which are not only differentially localized (11, 36-38) but also assume distinct metabolic roles within general isoprenoid biosynthetic pathways (39, 40). It would not be surprising to identify yet another variation of the MVA pathway in plants or an essential recycling route to IPP (1). The diversity of primary and secondary isoprenoid products produced by plants appear to be localized in subcellular compartments and organelles. A functional and ubiquitous IPK in the plant kingdom would afford distinct metabolic routes and fluxes to discrete pools of isoprenoid diphosphate substrates destined for specific primary or specialized plant metabolites.

These results biochemically elucidate the terminal biosynthetic steps in an alternative or unconventional MVA pathway. The experiments described also definitely demonstrate metabolic routes to IPP (1) through the classical MVA pathway in previously overlooked organisms by including the kinetic characterization of IPKs, MDDs, MPDs, and PMKs taken from all three domains of life. Significantly, the studies presented provide the first experimental support for the existence of a functioning alternative MVA pathway in the photosynthetic heterotrophic bacterial class Chloroflexi.

Experimental Procedures

Cloning of IPK Homologs.

Archaeal IPKs from M. jannaschii, M. maripaludis C5, and S. solfataricus P2 in addition to MDD and PMK from S. solfataricus P2 were cloned from genomic DNA from American Type Cell Cultures (ATCC) as previously described for M. jannaschii (18) into a pET28a(+) vector containing a thrombin-cleavable N-terminal 8-His tag. IPKs, MDDs, and PMKs from A. thaliana, T. adhaerens, B. floridae, and R. castenholzii were ordered as synthetic genes from Genscript (Piscataway, N.J., USA) and sub-cloned using Gateway technology from Invitrogen (San Diego, Calif., USA) into pHIS9GW, an in-house pet28-based vector modified to contain a thrombin-cleavable 9-His tag.

Protein Expression and Purification.

All proteins were expressed according to a previously described procedure with several modifications (18). Generally, each plasmid containing the gene of interest was transformed into B121 (DE3) cells (Novagen), grown at 37° C. in 1 L cultures of TB media to an OD₆₀₀ₙₘ of 1.0, induced with 1 mM IPTG, and grown overnight at 20° C. All proteins were purified similarly and as previously described (18); however, only the M. jannaschii protein was incubated at 80° C. during purification.

Steady-state kinetic analyses. Kinetic measurements were performed on IPKs from M. maripaludis, S. solfataricus, and B. floridae using a coupled pyruvate kinase-lactate dehydrogenase assay as previously described that employs varying IP concentrations ranging from 2 μM-1 mM (18). The substrate IP was purchased from Isoprenoids, LC (>95% purity). Steady-state kinetic curves were fitted using Prism (GraphPad Software Inc., San Diego, Calif., USA) to compute K_{M}, k_{cat}, and where appropriate, K. Activity measurements were performed for T. adhaerens and A. thaliana using the coupled assay at four different IP concentrations (2 μM, 10 μM, 50 μM, and 100 μM) in triplicate. Kinetic measurements were performed on MDDs from B. floridae and A. thaliana as discussed above with concentrations of (RS)-MVAPP (Sigma, 95% purity) ranging from 2 μM-1 mM. Kinetic measurements were performed on MPD from R. castenholzii and PMKs from B. floridae, A. thaliana, and S. solfataricus with concentrations of (RS)-MVAP (Sigma, 95% purity) ranging from 5 μM-2.5 mM.

GC-MS Assays.

All GC-MS assays were carried out in two steps. First, 1 μM of each of two enzymes (PMK and MDD for the classical MVA pathway, MDD and IPK for the alternative MVA pathway) was incubated for 30 min with 500 μM (RS)-MVAP, 4 mM ATP, and 10 mM MgCl₂ buffered with 50 mM Tris-HCl, pH 8.0. Second, 40 μl of this reaction was transferred to a glass vial containing 10 mM MgCl₂ buffered with 50 mM Tris-HCl, pH 8.0, containing at least a 150-fold excess of each of the following enzymes: E. coli isopentenyl diphosphate isomerase (IPPI), E. coli farnesyl diphosphate synthase (FPPS), and tobacco 5-epi-aristolochene synthase (TEAS). This reaction mixture was overlaid with ethyl acetate, incubated overnight, and vortexed to extract hydrocarbons from the aqueous layer. GC-MS assays were performed as previously described (18). All values were compared to a control reaction, where FPP was added in place of MVA pathway enzymes at an appropriate concentration to simulate complete turnover of MVAP.

Bioinformatic Analyses on IPK Homologs.

Public protein, cDNA, EST and genomic databases were searched for IPK homologs, using individual IPK protein sequences, and profile Hidden Markov models built from several individual IPK clades. Genes were predicted from genomic sequences using Genewise (19) and TimeLogic® GeneDetective™ (Active Motif Inc., Carlsbad, Calif.) programs, with manual editing. Protein sequences were aligned with Muscle (20) and edited with ClustalX (21) and JalView (22). FIG. 2 was created using PhyML (23) using the SPR model and rooted with fosfomycin kinase sequences. Manual editing was used to merge EST sequences and gene predictions, to correct frameshifts, and to fuse one gene split across two contigs. Discrepancies between individual ESTs were resolved to maximize sequence similarity to close homologs. IPK homologs from archaea were found in all but three of the 74 complete archaeal genomes found in the Integrated Microbial Genomes (IMG) database as of Mar. 8, 2010(24). The exceptions are S. acidocaldarius and S. tokodaii, and Nanoarchaeum equitans, a symbiont archaeon with a reduced genome. Within bacteria, clear IPK homologs were found only in all five sequenced genomes of the class Chloroflexi, but not within other classes of the phylum Chloroflexi. Divergent homologs were found in Streptomyces wedmorensis, Streptomyces fradiae and one strain of Pseudomonas syringae (all probably fosfomycin kinases), and Shewanella denitrificans. The P. syringae gene is found only in a contig from strain PB-5123, and not several other sequenced strains. The sequence contains a frameshift within the ORF and lacks the H60 residue, both of which may be sequencing errors.

In eukaryotes, searches for IPK homologs were made of the non-redundant amino acid (NRAA) Genbank database (25), the database of expressed sequence tags (dbEST) (26), and a wide variety of genome databases, including those at Ensembl (www.ensembl.org) (27), Joint Genome Institute (JGI, genome.jgi-psf.org/), Baylor College of Medicine (www.hgsc.bcm.tmc.edu), Sanger Institute (www.genedb.org/) and the Broad Institute (www.broadinstitute.org). Searches were with a series of IPK homologs (blastp against predicted peptides, tblastn against genome) and using a hidden Markov model profile searched against the genome, using Gene Detective.

MDD Alignments.

Alignments of MDDs were performed using a combined structure and sequence-based approach. Representatives from each of four groups (eukaryotes, archaea, bacteria, and Chloroflexi) were modeled using Swissmodel and superimposed to identify aligning active site residues (see Table 3). Each of these four groups were then aligned with other sequences from the same group using the programs Muscle (20) and Jalview (22) to generate four separate alignments.

### III. Tables

TABLE 1
Highly Conserved Residues in IPK

| Highly-conserved residues^{a} | Partially conserved residues |
|---|---|
| K6, G8, G9, K15, G54, H60, P140, G144, | K221^{b}, G253^{c}, T254^{c} |
| D213, T215, G216 | |

^{a}numbering is in accordance with IPK from M. jannaschii
^{b}conserved in all but one possible gene prediction error
^{c}may also be invariant, but mis-predicted in several sequences.

TABLE 2
Kinetic Constants for Characterized Enzymes of the MVA pathway

| Organism | gi number | Enzyme | Substrate | K_{M} (μM) | k_{cat} (s⁻¹) | Kᵢ (μM) | k_{cat}/K_{M} (s⁻¹μM⁻¹) | R² (^{a}) |
|---|---|---|---|---|---|---|---|---|
| M. jannaschii | 15668214 | IPK | IP | 4.3 (±0.6)^{b} | 1.46 (±0.03) | —^{c} | 0.34 (±0.05) | 0.90 |
| M. maripaludis | 132664414 | IPK | IP | 21.4 (±4.3) | 15.2 (±1.4) | 877 (±550) | 0.71 (±0.16) | 0.99 |
| S. solfataricus | 15897030 | IPK | IP | 23.6 (±4.8) | 0.91 (±0.05) | — | 0.04 (±0.01) | 0.92 |
| | 15899698 | PMK | MVAP | 23.1 (±3.8) | 1.98 (±0.10) | 5500 (±2200) | 0.09 (±0.01) | 0.98 |
| R. castenholzii | 156743980 | IPK | IP | 4.3 (±0.7) | 1.70 (±0.04) | — | 0.40 (±0.06) | 0.96 |
| | 156740939 | MPD | MVAP | 1150 (±203) | 0.94 (±0.08) | — | 0.0008 (±0.0001) | 0.98 |
| | (SEQ ID NO: 1) | | | | | | | |
| | 156740939 | MDD | MVAPP | ND^{d} | ND | ND | ND | ND |
| B. floridae | NA^{e} | IPK | IP | 13.3 (±2.0) | 27.2 (±1.2) | 2820 (±1700) | 2.05 (±0.32) | 0.98 |
| | 260829481 | PMK | MVAP | 38.0 (±7.3) | 12.6 (±0.4) | — | 0.33 (±0.06) | 0.93 |
| | 260794527 | MDD | MVAPP | 15.1 (±3.7) | 1.36 (±0.09) | — | 0.09 (±0.02) | 0.89 |
| A. thaliana | 22329798 | IPK | IP | 0.79 (±0.35) | 1.9 (±0.2) | 522 (±381) | 2.4 (±1.1) | 0.95 |
| | 15222502 | PMK | MVAP | 11.8 (±2.0) | 20.9 (±0.7) | — | 1.77 (±0.31) | 0.97 |
| | 15224931 | MDD | MVAPP | 15.7 (±5.0) | 2.02 (±0.13) | — | 0.13 (±0.04) | 0.89 |
| T. adhaerens | 195996013 | IPK | IP | 3.1 (±1.6) | 2.4 (±0.2) | — | 0.77 (0.40) | 0.79 |

^{a}R² represents goodness of fit for the kinetic curve to the experimental data. R² = 1.0 − (SS_{reg}/SSₜₒₜ), where SS_{reg} = sum of squares value, SSₜₒₜ = sum of squares of the distances between each point and a horizontal line passing through the average of all y values
^{b}Values in parentheses represent standard error (or propagation of error) for each kinetic constant
^{c}Kᵢ constant was not calculated or was not applicable
^{d}Not detected
^{e}B. floridae IPK sequence was predicted from scaffold_167 of the genome assembly(41) using genewise and manual annotation

TABLE 3
Active site phosphate binding residues commonly observed in
MDDs across the three domains of life
MDD-like active site residues

| | | | | | | |
|---|---|---|---|---|---|---|
| S. epidermis | Tyr18 | Lys21 | Ile27 | Ser139 | Ser141 | Ser192 |
| Bacteria | Tyr | Lys | variable^{a} | Ser | Ser | Ser |
| Sulfolobales | Tyr | Lys | Asn | Ser | Ser | Ser |
| Eukarya | Tyr | Lys | Ile/Asn | Ser | Ser | Ser |

MPD-like active site residues

| | | | | | | |
|---|---|---|---|---|---|---|
| R. | Tyr74 | Leu77 | Arg83 | Ala201 | Ser203 | Thr255 |
| castenholzii | | | | | | |
| Haloarchaea/ | Tyr/Phe | Met/Tyr | Arg | Ser | Ser | polar^{b} |
| Thermo- | | | | | | |
| plasmatales | | | | | | |
| Chloroflexi | Tyr | Leu | Arg/Thr | Ala | Ser | Thr |

^{a}Not conserved among canonical bacterial MDDs.
^{b}Includes Glu, Asp, Asn, and Ser

### IV. References

1. Lu S & Li L (2008) Carotenoid metabolism: biosynthesis, regulation, and beyond. Integr Plant Biol 50(7):778-785.
2. Hedden P & Kamiya Y (1997) Gibberellin Biosynthesis: Enzymes, Genes and Their Regulation. Annu Rev Plant Physiol Plant Mol Biol 48:431-460.
3. Nowicka B & Kruk J (Occurrence, biosynthesis and function of isoprenoid quinones. Biochim Biophys Acta 1797(9):1587-1605.
4. Chew A G & Bryant D A (2007) Chlorophyll biosynthesis in bacteria: the origins of structural and functional diversity. Annu Rev Microbiol 61:113-129.
5. Schafer W R & Rine J (1992) Protein prenylation: genes, enzymes, targets, and functions. Annu Rev Genet. 26:209-237.
6. Lynen F (1967) Biosynthetic pathways from acetate to natural products. Pure Appl Chem 14(1):137-167.
7. Katsuki H & Bloch K (1967) Studies on the biosynthesis of ergosterol in yeast. Formation of methylated intermediates. J Biol Chem 242(2):222-227.
8. Arigoni D, et al. (1997) Terpenoid biosynthesis from 1-deoxy-D-xylulose in higher plants by intramolecular skeletal rearrangement. Proc Natl Acad Sci USA 94(20):10600-10605.
9. Rohmer M (1999) The discovery of a mevalonate-independent pathway for isoprenoid biosynthesis in bacteria, algae and higher plants. Nat Prod Rep 16(5):565-574.
10. Eisenreich W, et al. (1998) The deoxyxylulose phosphate pathway of terpenoid biosynthesis in plants and microorganisms. Chem Biol 5(9):R221-233.
11. Lange B M, Rujan T, Martin W, & Croteau R (2000) Isoprenoid biosynthesis: the evolution of two ancient and distinct pathways across genomes. Proc Natl Acad Sci USA 97(24):13172-13177.
12. Cassera M B, et al. (2004) The methylerythritol phosphate pathway is functionally active in all intraerythrocytic stages of Plasmodium falciparum. J Biol Chem 279(50):51749-51759.
13. Bochar D A, Stauffacher C V, & Rodwell V W (1999) Sequence comparisons reveal two classes of 3-hydroxy-3-methylglutaryl coenzyme A reductase. Mol Genet Metab 66(2):122-127.
14. Lombard J & Moreira D (2010) Origins and early evolution of the mevalonate pathway of isoprenoid biosynthesis in the three domains of life. Mol Biol Evol.
15. Matsumi R, Atomi H, Driessen A J, & van der Oost J (2010) Isoprenoid biosynthesis in Archaea—biochemical and evolutionary implications. Res Microbiol 162(1):39-52.
16. Grochowski L L, Xu H, & White R H (2006) Methanocaldococcus jannaschii uses a modified mevalonate pathway for biosynthesis of isopentenyl diphosphate. J Bacteriol 188(9):3192-3198.
17. Miziorko H M (2010) Enzymes of the mevalonate pathway of isoprenoid biosynthesis. Arch Biochem Biophys 505(2):131-143.
18. Dellas N & Noel J P (2010) Mutation of archaeal isopentenyl phosphate kinase highlights mechanism and guides phosphorylation of additional isoprenoid monophosphates. ACS Chem Biol 5(6):589-601.
19. Birney E, Clamp M, & Durbin R (2004) GeneWise and Genomewise. Genome Res 14(5):988-995.
20. Edgar R C (2004) MUSCLE: multiple sequence alignment with high accuracy and high throughput. Nucleic Acids Res 32(5):1792-1797.
21. Larkin M A, et al. (2007) Clustal W and Clustal X version 2.0. Bioinformatics 23(21):2947-2948.
22. Waterhouse A M, Procter J B, Martin D M, Clamp M, & Barton G J (2009) Jalview Version 2-a multiple sequence alignment editor and analysis workbench. Bioinformatics 25(9):1189-1191.
23. Guindon S, Lethiec F, Duroux P, & Gascuel O (2005) PHYML Online—a web server for fast maximum likelihood-based phylogenetic inference. Nucleic Acids Res 33(Web Server issue):W557-559.
24. Markowitz V M, et al. (2008) The integrated microbial genomes (IMG) system in 2007: data content and analysis tool extensions. Nucleic Acids Res 36(Database issue):D528-533
25. Benson D A, Karsch-Mizrachi I, Lipman D J, Ostell J, & Sayers E W (2010) GenBank. Nucleic Acids Res 38(Database issue):D46-51.
26. Boguski M S, LoApplicants™, & Tolstoshev C M (1993) dbEST—database for “expressed sequence tags”. Nat Genet. 4(4):332-333.
27. Birney E, et al. (2004) Ensembl 2004. Nucleic Acids Res 32(Database issue):D468-470.
28. Arnold K, Bordoli L, Kopp J, & Schwede T (2006) The SWISS-MODEL workspace: a web-based environment for protein structure homology modelling. Bioinformatics 22(2):195-201.
29. Kiefer F, Arnold K, Kunzli M, Bordoli L, & Schwede T (2009) The SWISS-MODEL Repository and associated resources. Nucleic Acids Res 37(Database issue):D387-392.
30. Byres E, Alphey M S, Smith T K, & Hunter W N (2007) Crystal structures of Trypanosoma brucei and Staphylococcus aureus mevalonate diphosphate decarboxylase inform on the determinants of specificity and reactivity. J Mol Biol 371(2):540-553.
31. Barta M L, et al. (2011) Crystal Structures of Staphylococcus epidermidis Mevalonate Diphosphate Decarboxylase Bound to Inhibitory Analogs Reveal New Insight into Substrate Binding and Catalysis. J Biol Chem 286(27):23900-23910.
32. Mulkidjanian A Y, et al. (2006) The cyanobacterial genome core and the origin of photosynthesis. Proc Natl Acad Sci USA 103(35):13126-13131.
33. Hohmann-Marriott M F & Blankenship R E (2011) Evolution of photosynthesis. Annu Rev Plant Biol 62:515-548.
34. Bryant D A & Frigaard N U (2006) Prokaryotic photosynthesis and phototrophy illuminated. Trends Microbiol 14(11):488-496.
35. Nishimura H, et al. (2013) Biochemical evidence supporting the presence of the classical mevalonate pathway in the thermoacidophilic archaeon Sulfolobus solfataricus. Journal of biochemistry.
36. Hsieh M H, Chang C Y, Hsu S J, & Chen J J (2008) Chloroplast localization of methylerythritol 4-phosphate pathway enzymes and regulation of mitochondrial genes in ispD and ispE albino mutants in Arabidopsis. Plant Mol Biol 66(6):663-673.
37. Leivar P, et al. (2005) Subcellular localization of Arabidopsis 3-hydroxy-3-methylglutaryl-coenzyme A reductase. Plant Physiol 137(1):57-69.
38. Nagegowda D A, Ramalingam S, Hemmerlin A, Bach T J, & Chye M L (2005) Brassica juncea HMG-CoA synthase: localization of mRNA and protein. Planta 221(6):844-856.
39. Nes W D & Venkatramesh M (1999) Enzymology of phytosterol transformations. Crit. Rev Biochem Mol Biol 34(2):81-93.
40. Eisenreich W, Rohdich F, & Bacher A (2001) Deoxyxylulose phosphate pathway to terpenoids. Trends Plant Sci 6(2):78-84.
41. Putnam N H, et al. (2008) The amphioxus genome and the evolution of the chordate karyotype. Nature 453(7198):1064-1063

What is claimed is:

1. A method of synthesizing an isopentyl phosphate or analog thereof, said method comprising contacting a phosphomevalonate (MVAP) or analog thereof with a mevalonatephosphate decarboxylase (MPD)-like mevalonate 5-diphosphate decarboxylase (MDD) enzyme, thereby forming an isopentyl phosphate or analog thereof, wherein said MPD-like MDD enzyme lacks detectable MDD activity.

2. The method of claim 1, wherein said MPD-like MDD enzyme does not accept diphosphomevalonate (MVAPP) as a substrate.

3. The method of claim 1, wherein said MPD-like MDD enzyme is at least 50, 100, 150, 200, 210, 220, 230, 240, 250, 252, 254, 256, 258, 259 or 260 amino acids in length.

4. The method of claim 1, wherein said MPD-like MDD enzyme comprises the amino acid sequence as set forth by SEQ ID NO:1.

5. The method of claim 1, wherein said MPD-like MDD enzyme comprises amino acids selected from the group consisting of Lys16, Tyr74, Leu77, Arg83, Ala201, Ser203 and Thr255.

6. A method of synthesizing an isopentyl diphosphate or analog thereof, said method comprising:
(i) contacting a phosphomevalonate (MVAP) or analog thereof with a mevalonatephosphate decarboxylase (MPD)-like mevalonate 5-diphosphate decarboxylase (MDD) enzyme, thereby forming an isopentyl phosphate or analog thereof; and
(ii) contacting said isopentyl phosphate or analog thereof and a phosphate or phosphate analog donor with an isopentenyl phosphate kinase or an mevalonate 5-diphosphate decarboxylase, thereby forming an isopentyl diphosphate or analog thereof.

7. A method of identifying an amino acid substitution within a mevalonatephosphate decarboxylase (MPD)-like mevalonate 5-diphosphate decarboxylase (MDD) enzyme that increases isopentyl phosphate formation rate, said method comprising:
(i) determining a hypothetical binding position of a phosphomevalonate (MVAP) within an active site of a first MPD-like MDD enzyme using a computer modeling program;
(ii) based on the hypothetical binding position, making a test mutated MPD-like MDD enzyme comprising an amino acid substitution relative to said first MPD-like MDD enzyme;
(iii) contacting said test mutated MPD-like MDD enzyme with an MVAP and determining a first rate of formation of an isopentyl phosphate;
(iv) comparing the first rate of formation of said isopentyl phosphate with a second rate of formation, wherein said second rate of formation is determined by contacting said first MPD-like MDD enzyme and said MVAP, wherein a higher first rate of formation relative to the second rate of formation indicates said amino acid substitution increases isopentyl phosphate formation rate.

8. The method of claim 7, wherein said MVAP comprises a detectable label.

9. The method of claim 8, wherein said detectable label is selected from the group consisting of fluorescent label, luminescent label, radioactive label, spectroscopic label, stable isotope mass tagged label, electron spin resonance label, nuclear magnetic resonance label and chelated metal label.
